(19) United States
(12) Patent Application Publication
(10) Pub. No.: US 2011/0230561 A1
(43) Pub. Date: Sep. 22, 2011

(54) MEDICAL ADHESIVE COMPOSITIONS

(76) Inventors: Hongbo Liu, Hillsborough, NJ (US); Sandra Gonzalez, Bedminster, NJ (US); Breda Mary Cullen, North Yorkshire (GB); Derek Walter Silcock, North Yorkshire (GB)

(21) Appl. No.: 13/130,591

(22) PCT Filed: Nov. 17, 2009
(86) PCT No.: PCT/US2009/064796

§ 371 Date: May 23, 2011

(30) Foreign Application Priority Data
Nov. 25, 2008 (GB) 0821532.9

(51) Int. Cl. A61K 47/16; A61K 31/192; A61K 31/16; C08F 120/44; A61P 29/00

(52) U.S. Cl. 514/570; 514/785; 514/630; 526/328

(57) ABSTRACT

A medical adhesive composition comprising, based upon the total weight of the composition, from about 50 wt. % to about 99.9 wt. % of one or more α-cyanoacrylate monomers and from about 0.1 wt. % to about 5 wt. % of one or more non-steroidal anti-inflammatory drugs (NSAIDs). Suitable NSAIDs include ibuprofen and acetaminophen. The resulting compositions provide enhances fibroblast proliferation and reduced cytotoxicity compared to compositions that do not contain NSAID.

All patent documents referred to herein are incorporated by reference in their entirety.

The present invention relates to medical adhesive and sealant compositions, comprising an α-cyanoacrylate monomer and a non-steroidal anti-inflammatory drug (NSAID), and to the medical uses thereof.

It is known that monomeric forms of α-cyanoacrylates are extremely reactive, polymerizing rapidly in the presence of even minute amounts of an initiator, including moisture present in the air or on moist surfaces such as animal tissue. Monomers of α-cyanoacrylates are anionically polymerizable or free radical polymerizable, or polymerizable by zwitterions or ion pairs to form polymers. Once polymerization has been initiated, the cure rate can be very rapid.

Since the discovery of the adhesive properties of α-cyanoacrylates monomers and polymers, they have found wide use due to the speed with which they cure, the strength of the resulting bond formed, and their relative ease of use. These characteristics have made α-cyanoacrylate adhesives the primary choice for numerous applications such as bonding plastics, rubbers, glass, metals, wood, and, more recently, biological tissues.

Medical applications of cyanoacrylate adhesive compositions include use as an alternate or an adjunct to surgical sutures and staples in wound closure as well as for covering and protecting surface wounds such as lacerations, abrasions, burns, stomatitis, sores, and other surface wounds. When an adhesive is applied, it is usually applied in its monomeric form, and the resultant polymerization gives rise to the desired adhesive bond.

For example, polymerizable cyanoacrylates, and medical adhesive compositions comprising such monomers, are disclosed in U.S. Pat. No. 5,328,687.

It is known to use cyanoacrylate adhesives to deliver bioactive agents to a wound site. For example, U.S. Pat. No. 5,582,834, U.S. Pat. No. 5,575,997, and U.S. Pat. No. 5,624,669 disclose such technology. Examples of such bioactive agents include antimicrobial agents to be released into the wound. For example, EP-A-1508601 describes cyanoacrylate medical adhesive compositions containing one or more phenolic antimicrobial agents, suitably Triclosan. U.S. Pat. No. 7,238,828 describes cyanoacrylate medical adhesives optionally containing a wide range of possible bioactive agents.

However, a drawback to the in vivo biomedical use of α-cyanoacrylate monomers and polymers has been their potential for causing adverse tissue response. For example, methyl α-cyanoacrylate has been reported to cause tissue inflammation at the site of application. It has further been found that α-cyanoacrylates are cytotoxic in use, in particular that they cause significant cell death to mammalian fibroblasts. This cytotoxicity may interfere with normal wound healing.

It has been suggested that the adverse tissue response to α-cyanoacrylates may be caused by the products released during in vivo biodegradation of the polymerized α-cyanoacrylates. It has been suggested that formaldehyde is the biodegradation product most responsible for the adverse tissue response and, specifically, the high concentration of formaldehyde produced during rapid polymer biodegradation.

Efforts to increase the tissue compatibility of α-cyanoacrylates have included modifying the alkyl ester group. For example, increasing the alkyl ester chain length to form the higher cyanoacrylate analogues, e.g., butyl-2-cyanoacrylates and octyl-2-cyanoacrylates, has been found to improve biocompatibility but the higher analogues biodegrade at slower rates than the lower alkyl cyanoacrylates.

Other efforts to increase the tissue compatibility of α-cyanoacrylates included the addition of a formaldehyde scavenger compound. U.S. Pat. No. 5,328,687 and U.S. Pat. No. 5,624,669 set forth various formaldehyde scavenger compounds suitable for use in medical adhesive composition, including sulfites; bisulfites; mixtures of sulfites and bisulfites; ammonium sulfite salts; amines; amides; imides; nitriles; carbamates; alcohols; mercaptans; proteins; mixtures of amines, amides, and proteins; active methylene compounds such as cyclic ketones and compounds having a β-dicarbonyl group; and certain heterocyclic ring compounds free of a carbonyl group and containing an NH group.

However, a need remains for improved α-cyanoacrylate monomer medical adhesive compositions that are less cytotoxic in use, but where the performance of the adhesive composition is not compromised.

In a first aspect, the present invention provides a medical adhesive composition comprising, based upon the total weight of the composition, from about 50 wt. % to about 99.9 wt. % of one or more α-cyanoacrylate monomers and from about 0.1 wt. % to about 5 wt. % of one or more non-steroidal anti-inflammatory drugs (NSAIDs).

In a second aspect, the present invention provides a kit comprising a first container that contains a sterile medical adhesive composition according to the first aspect of the invention sealed therein and a second container that contains a polymerization initiator or accelerator.

In a further aspect, the present invention provides a medical adhesive polymer obtainable by polymerizing a medical adhesive composition according to the first aspect of the invention.

The present inventors have found that the adhesive compositions containing NSAIDs are significantly less cytotoxic, in particular with respect to fibroblasts, than corresponding compositions without NSAIDs. This cytoprotective effect of NSAIDs was unexpected and unpredictable. Without wishing to be bound by any theory, it is thought that the NSAIDs may be reacting with certain breakdown products of the cyanoacrylate adhesive, or may be otherwise inhibiting the cytotoxic effect of the said breakdown products.

Accordingly, in a further aspect the present invention provides a method of enhancing fibroblast cell viability in mammalian tissue in contact with an α-cyanoacrylate adhesive formed by polymerization of an α-cyanoacrylate adhesive composition applied to said tissue, said method comprising dispersing from about 0.1 wt. % to about 5 wt. %, based upon the weight of said composition, of one or more non-steroidal anti-inflammatory drugs (NSAIDs) in said composition prior to application of said composition to said tissue.

In a further aspect the present invention provides a method of reducing cytotoxicity of an α-cyanoacrylate adhesive formed by polymerization of an α-cyanoacrylate adhesive composition, said method comprising dispersing from about 0.1 wt. % to about 5 wt. %, based upon the weight of said composition, of one or more non-steroidal anti-inflammatory drugs (NSAIDs) in said α-composition prior to application of said composition to said tissue.

In a further aspect the present invention provides the use of a non-steroidal anti-inflammatory drug (NSAID) for the preparation of a medical adhesive composition comprising an α-cyanoacrylate monomer or polymer and, based upon the total weight of the composition, from about 0.1 wt. % to about 5 wt. % of said NSAID for enhancing fibroblast cell viability in a mammalian tissue in contact with said medical adhesive.

In a further aspect the present invention provides the use of a non-steroidal anti-inflammatory drug (NSAID) for the preparation of a medical adhesive composition comprising an α-cyanoacrylate monomer or polymer and, based upon the total weight of the composition, from about 0.1 wt. % to about 5 wt. % of said NSAID for reducing cytotoxicity of said medical adhesive composition.

Any known NSAID is suitable for use as the NSAID component of compositions according to the present invention. The NSAIDs generally share the common functional feature that they are inhibitors of cyclooxygenase enzymes (Cox-1 and/or Cox-2). Possible NSAIDS include, but are not limited to:

(a) the salicylates, such as aspirin;

(b) the propionic acids, or profens, such as carprofen, fenoprofen, flurbiprofen, ibuprofen, ketoprofen, naproxen, pranoprofen and suprofen;

(c) the acetic acid derivatives such as diclofenac, etodolac, ibufenac, indomethacin, sulindac, tolmetin and zomepirac;

(d) the biphenylylcarboxylic acids such as diflunisal and flufenisal; and

(e) the p-amidophenol compounds, such as acetaminophen.

Suitable NSAIDs for the practice of the present invention include ibuprofen, acetaminophen, Ketoprofen, Topiramate, Curcumin, and mixtures thereof. Especially suitable NSAIDs for the practice of the present invention include ibuprofen, acetaminophen, and mixtures thereof.

The amount of NSAID that is added to the monomer composition depends upon several factors, including, but not limited to, the specific NSAID being used, the amount of the NSAID suitable for use in the compositions, and whether and to what extent the NSAID is regulated by the U.S. FDA (or other appropriate regulatory agencies or bodies of the United States or foreign countries). As already noted, the compositions according to the invention comprise, based upon the total weight of the compositions, from about 0.1 wt. % to about 5 wt. % of the one or more NSAIDs. Suitably, the compositions comprise from about 0.2 wt. % to about 4 wt. %, for example from about 0.5 wt. % to about 2 wt. % of the one or more NSAIDs.

In embodiments, the NSAID is soluble in the adhesive composition at room temperature and the resultant composition is stable for at least a given amount of time. However, in some specific embodiments, complete solubility may not be required. Production of the composition includes mixing the polymerizable monomers and the NSAID in a container, and in one embodiment for a period of time until the mixture is visually homogenous.

Suitably, the NSAIDs are stable in the monomer composition (i.e., do not cause premature polymerization), and do not affect the polymerization rate of the composition by initiating or inhibiting polymerization. Although some change in the polymerization rate may occur, suitably the NSAID does not substantially affect the polymerization rate of the monomer. For example, the polymerization rate of the monomer composition with the NSAID should differ from the polymerization rate of a comparable monomer composition without the NSAID by no more than about 50%, suitably no more than about 20%.

The polymerizable monomer, and the composition as a whole, is suitably in liquid or gel form at ambient temperatures (20-25° C.).

The adhesive composition may be sterilized via any known method for sterilizing cyanoacrylates. Production of the sterilized composition includes placing the polymerizable monomers and the NSAID in a container, sealing the container and sterilizing the container and the mixture. The NSAID in combination with the monomer composition should be compatible with one or more sterilization procedures. Suitably, the NSAID is compatible with sterilization processing of said composition.

In embodiments of the present invention, the NSAID exhibits stability in the monomer composition for at least five minutes after mixing or dissolving the agent in the polymerizable monomer compound, and/or sterilizing a resultant combination. In one embodiment, the NSAID is soluble in said monomer at room temperature and substantially all of said monomer remains stable for at least five minutes after forming the composition.

More suitably, stability of the adhesive composition is maintained for at least one hour, suitably ten hours, and more suitably twenty-four hours after mixing the NSAID with the polymerizable monomer compound, and/or sterilizing a resultant combination. Suitably, the adhesive composition remains stable for at least one hour, more suitably for at least twenty-four hours, after forming the composition. Even more suitably, stability of the adhesive composition is maintained for a time period sufficient to provide a commercially significant shelf-life to the adhesive composition, or even an extended shelf-life as compared to similar compositions not including the NSAID. Suitably, the composition remains stable for at least eighteen months after forming the composition. As used herein, “stability” refers to the resultant composition maintaining a commercially acceptable form for the prescribed amount of time. That is, the composition does not prematurely polymerize or otherwise change form or degrade to the point that the composition is not useful for its intended purpose. Thus, while some polymerization or thickening of the composition may occur, such as can be measured by changes in viscosity of the composition, such change is not so extensive as to destroy or significantly impair the usefulness of the composition.

In embodiments, the adhesive composition has a viscosity of about 1 centipoise to about 5000 centipoise, such as about 3 centipoise to about 600 centipoise, or about 5 centipoise to about 40 centipoise. The viscosity can be selected according to the proposed use, e.g. 4-50 centipoise for certain uses and about 100 centipoise to about 250 centipoise for other uses. Additionally, the composition may be a gel, e.g., about 50,000 centipoise to about 500,000 centipoise. A gel is a combination of a disperse phase with a continuous phase to produce a semisolid material. The viscosity of the adhesive composition may be measured with a Brookfield Viscometer at 25° C. Additionally, in embodiments where a sterilization treatment is applied, the viscosity of the composition should suitably be maintained or increased by a controlled and acceptable amount after sterilization.

Typically, for medical purposes, an adhesive should have a shelf-life of at least one year; however, an increased shelf-life beyond this provides increased economic advantages to both the manufacturer and the consumer. As used herein, shelf-life refers to the amount of time the container and composition therein can be held at ambient conditions (approximately room temperature) or less, without degradation of the composition and/or container occurring to the extent that the composition and container cannot be used in the manner and for the purpose for which they were intended. Thus, while some degradation to either or both of the composition and container can occur, it must not be to such an extent that the composition and/or container is no longer useable. As used herein, an “extended shelf-life” refers to a shelf-life of at least 12 months, suitably at least 18 months, more suitably at least 24 months, and even more suitably, at least 30 months.

In embodiments, the adhesive composition and/or its packaging can be sterilized. In a preferred embodiment, the composition is sterile. Furthermore, whether or not the composition and container are sterilized, the composition can further include one or more suitable preservatives, as described below.

Sterilization of the adhesive composition and/or its packaging can be accomplished by techniques known to the skilled artisan, and is suitably accomplished by methods including, but not limited to, chemical, physical, and/or irradiation methods. Examples of chemical methods include, but are not limited to, exposure to ethylene oxide or hydrogen peroxide vapor. Examples of physical methods include, but are not limited to, sterilization by heat (dry or moist) or retort canning. Examples of irradiation methods include, but are not limited to, gamma irradiation, electron beam irradiation, and microwave irradiation. Suitably, sterilizing is performed by dry heat, moist heat, gamma irradiation, electron beam irradiation (for example as described in U.S. Pat. No. 6,143,805.), microwave irradiation, or retort canning. The composition should also show low levels of toxicity to living tissue during its useful life. In one embodiment of the present invention, the composition is sterilized to provide a Sterility Assurance Level (SAL) of at least about 10⁻³ In embodiments, the Sterility Assurance Level may be at least about 10⁻⁴ or may be at least about 10⁻⁵, or may be at least about 10⁻⁶.

The adhesive composition includes a major fraction of one or more polymerizable α-cyanoacrylate monomers. Suitable monomers that may be used in this invention are readily polymerizable, e.g. anionically polymerizable or free radical polymerizable, or polymerizable by zwitterions or ion pairs to form polymers. Such monomers suitably include those that form polymers that are biodegradable in vivo. Such monomers are disclosed in, for example, U.S. Pat. No. 5,328,687, U.S. Pat. No. 5,928,611, U.S. Pat. No. 6,183,593, U.S. Pat. No. 6,183,593 and U.S. Pat. No. 7,238,828.

In one embodiment, the monomers include alkyl α-cyanoacrylates having an alkyl chain length of from about 1 to about 20 carbon atoms or more, suitably from about 3 to about 8 carbon atoms.

The α-cyanoacrylates useful in the compositions of the present invention can be prepared according to several methods known in the art. U.S. Pat. No. 2,721,858, U.S. Pat. No. 3,254,111, U.S. Pat. No. 3,995,641, and U.S. Pat. No. 4,364,876 disclose methods for preparing α-cyanoacrylates.

Suitable α-cyanoacrylate monomers used in this invention include methyl cyanoacrylate, ethyl cyanoacrylate, n-butyl cyanoacrylate, 2-octyl cyanoacrylate, methoxyethyl cyanoacrylate, ethoxyethyl cyanoacrylate, dodecyl cyanoacrylate, 2-ethylhexyl cyanoacrylate, butyl cyanoacrylate, 3-methoxybutyl cyanoacrylate, 2-butoxyethyl cyanoacrylate, 2-isopropoxyethyl cyanoacrylate, 1-methoxy-2-propyl cyanoacrylate, hexyl cyanoacrylate, or dodecylcyanoacrylate.

Other suitable cyanoacrylates for use in the present invention also include, but are not limited to, alkyl ester cyanoacrylate monomers such as those described in detail in EP-A-1317294. Examples of suitable alkyl ester cyanoacrylates include, but are not limited to, butyl lactoyl cyanoacrylate (BLCA), butyl glycoloyl cyanoacrylate (BGCA), ethyl lactoyl cyanoacrylate (ELCA), and ethyl glycoloyl cyanoacrylate (EGCA).

In certain embodiments, the medical adhesive composition according to the first aspect of the invention further comprises an antimicrobial agent. Suitable antimicrobial agents are described in EP-A-1508601. They include the various phenolic active compounds, and phenol derivatives, such as halogenated phenol compounds, including chlorinated or brominated phenol compounds. Suitable specific examples include, but are not limited to, tribromophenol, trichlorophenol, tetrachlorophenol, nitrophenol, 3-methyl-4-chloro-phenol, 3,5-dimethyl-4-chlorophenol, phenoxyethanol, dichlorophene, o-phenyl-phenol, m-phenylphenol, p-phenylphenol, 2-benzyl-4-chlorophenol, 2,4-dichloro-3,5-dimethylphenol, 4-chlorothymol, chlorphen, triclosan, fentichlor, phenol, 2-methyl phenol, 3-methyl phenol, 4-methyl phenol, 4-ethyl phenol, 2,4-dimethyl phenol, 2,5-dimethyl phenol, 3,4-dimethyl phenol, 2,6-dimethyl phenol, 4-n-propyl phenol, 4-n-butyl phenol, 4-n-amyl phenol, 4-tert-amyl phenol, 4-n-hexyl phenol, 4-n-heptyl phenol, and mono- and poly-alkyl and aromatic halophenols and their ammonium, alkali metal and alkaline earth metal salts, and mixtures thereof.

In embodiments, the antimicrobial agent is a halogenated phenol, such as a chlorinated phenol or a brominated phenol. Chlorinated phenol compounds that may be used according to the invention include but are not limited to parachlorometaxylenol, triclosan (2,4,4′-trichloro-2 hydroxy di-phenyl ether), p-chlorophenol, 2-chlorophenol, 3-chlorophenol, 4-chlorophenol, 2,4-dichlorophenol, 2,4,6-trichlorophenol, 2,3,4,6-tetrachlorophenol, pentachlorophenol, 4-chlororesorcinol, 4,6-dichlororesorcinol, 2,4,6-trichlororesorcinol, alkylchlorophenols (including p-alkyl-o-chlorophenols, o-alkyl-p-chlorophenols, dialkyl-4-chlorophenol, and tri-alkyl-4-chlorophenol), cyclohexyl p-chlorophenol, o-benzyl p-chlorophenol, o-benxyl-m-methyl p-chlorophenol, o-benzyl-m,m-dimethyl p-chlorophenol, o-phenylethyl p-chlorophenol, o-phenylethyl-m-methyl p-chlorophenol, dichloro-m-xylenol, chlorocresol, o-benzyl-p-chlorophenol, 3,4,6-trichlorphenol, 4-chloro-2-phenylphenol, 6-chloro-2-phenylphenol, o-benzyl-p-chlorophenol, 2,4-dichloro-3,5-diethylphenol, mixtures thereof, and the like. In one embodiment, the antimicrobial agent is a chlorinated phenol compound selected from the group consisting of parachlorometaxylenol, triclosan, p-chlorophenol, 2-chlorophenol, 3-chlorophenol, 4-chlorophenol, 2,4-dichlorophenol, 2,4,6-trichlorophenol, 2,3,4,6-tetrachlorophenol, pentachlorophenol, 4-chlororesorcinol, 4,6-dichlororesorcinol, 2,4,6-trichlororesorcinol, alkylchlorophenols, cyclohexyl p-chlorophenol, o-benzyl p-chlorophenol, o-benxyl-m-methyl p-chlorophenol, o-benzyl-m,m-dimethyl p-chlorophenol, o-phenylethyl p-chlorophenol, o-phenylethyl-m-methyl p-chlorophenol, dichloro-m-xylenol, chlorocresol, o-benzyl-p-chlorophenol, 3,4,6-trichlorphenol, 4-chloro-2-phenylphenol, 6-chloro-2-phenylphenol, o-benzyl-p-chlorophenol, 2,4-dichloro-3,5-diethylphenol, and mixtures thereof.

Specific examples of suitable alkyl chlorophenols include, but are not limited to, methyl p-chlorophenol, ethyl p-chlorophenol, n-propyl p-chlorophenol, n-butyl p-chlorophenol, n-amyl p-chlorophenol, sec-amyl p-chlorophenol, n-hexyl p-chlorophenol, n-heptyl p-chlorophenol, n-octyl p-chlorophenol, o-chlorophenol, methyl o-chlorophenol, ethyl o-chlorophenol, n-propyl o-chlorophenol, n-butyl o-chlorophenol, n-amyl o-chlorophenol, tert-amyl o-chlorophenol, n-hexyl o-chlorophenol, n-heptyl o-chlorophenol, 3-methyl p-chlorophenol, 3,5-dimethyl p-chlorophenol, 6-ethyl-3-methyl p-chlorophenol, 6-n-propyl-3-methyl p-chlorophenol, 6-iso-propyl-3-methyl p-chlorophenol, 2-ethyl-3,5-dimethyl p-chlorophenol, 6-sec-butyl-3-methyl p-chlorophenol, 2-iso-propyl-3,5-dimethyl p-chlorophenol, 6-diethylmethyl-3-methyl p-chlorophenol, 6-iso-propyl-2-ethyl-3-methyl p-chlorophenol, 2-sec-amyl-3,5-dimethyl p-chlorophenol, 2-diethylmethyl-3,5-dimethyl p-chlorophenol, 6-sec-octyl-3-methyl p-chlorophenol, 2,2′-methylene bis (4-chlorophenol), 2,2′-methylene bis (3,4,6-trichlorophenol), mixtures thereof, and the like. Brominated phenol compounds which may be used according to the invention include but are not limited to p-bromophenol, methyl p-bromophenol, ethyl p-bromophenol, n-propyl p-bromophenol, n-butyl p-bromophenol, n-amyl p-bromophenol, sec-amyl p-bromophenol, n-hexyl p-bromophenol, cyclohexyl p-bromophenol, o-bromophenol, tert-amyl o-bromophenol, n-hexyl o-bromophenol, n-propyl-m,m-dimethyl o-bromophenol, 2,2′-methylene bis (4-chloro-6-bromophenol), mixtures thereof, and the like. Suitably, the antimicrobial agent is a brominated phenol compound selected from the group consisting of p-bromophenol, methyl p-bromophenol, ethyl p-bromophenol, n-propyl p-bromophenol, n-butyl p-bromophenol, n-amyl p-bromophenol, sec-amyl p-bromophenol, n-hexyl p-bromophenol, cyclohexyl p-bromophenol, o-bromophenol, tert-amyl o-bromophenol, n-hexyl o-bromophenol, n-propyl-m,m-dimethyl o-bromophenol, 2,2′-methylene bis (4-chloro-6-bromophenol), and mixtures thereof.

Suitably, said antimicrobial agent comprises or consists essentially of Triclosan.

Suitably, the antimicrobial agent is present in the monomer composition in an amount such that the antimicrobial agent provides the desired antimicrobial effects at the application site. Without being bound by theory, it is believed that in one embodiment after the monomer composition is polymerized, the antimicrobial agent slowly elutes out of the polymer product over time. This slow elution of the anti-microbial agent enables the anti-microbial agent to be steadily released from the polymer product in order to provide the antimicrobial effect. Suitably, the antimicrobial agent is present in an amount of from about 0.001% to about 10%, more suitably from about 0.02% to about 2%, for example from about 0.1% to about 1%, by weight of the total composition.

The composition may optionally also include at least one plasticizing agent that assists in imparting flexibility to the polymer formed from the monomer. The plasticizing agent suitably contains little or no moisture and should not significantly affect the stability or polymerization of the monomer. Examples of suitable plasticizers include but are not limited to tributyl citrate, acetyl tri-n-butyl citrate (ATBC), polymethylmethacrylate, silicone oils, siloxanes, and others as listed in U.S. Pat. No. 6,183,593. Specific examples of the silicone oils and siloxanes include, for example, but are not limited to, polydimethylsiloxane, hexadimethylsilazane. Suitably, said plasticising agent is present in the composition in an amount of between about 5 wt. % and about 30 wt % based on the total composition, more suitably between about 10 wt % and about 25 wt %, for example about 10 wt % to about 20 wt %.

The composition may also optionally include at least one thixotropic agent. Suitable thixotropic agents are known to the skilled artisan and include, but are not limited to, silica gels such as those treated with a silyl isocyanate, and optionally surface treated titanium dioxide. Organic thixotropic agents such as polyvalent hydroxy compound-aromatic aldehyde condensate, aromatic hydroxy compound-boric acid semi-polar condensate, aluminum fatty acid salt, hydrogenated castor oil compound, and fatty acid polyamide compounds may be used, for example in amounts of about 0.1 parts to about 30 parts by weight per 100 parts of cyanoacrylate monomer. Examples of suitable thixotropic agents and thickeners are disclosed in, for example, U.S. Pat. No. 4,720,513, and U.S. Pat. No. 6,310,166.

The composition may optionally also include thickeners. Suitable thickeners may include poly (2-ethylhexyl methacrylate), poly(2-ethylhexyl acrylate) and others as listed in U.S. Pat. No. 6,183,593. The amount of thickening agent that is added to the monomer composition depends upon, for example, the molecular weight of the thickening agent and the desired characteristics of the composition. In one embodiment, the thickening agent may comprise from about 0.5 wt. % to about 25 wt % based on the weight of the adhesive composition, for example from about 1 wt % to about 10 wt %, typically from about 1 wt. % to about 5 wt %, of the adhesive composition. In some embodiments, the thickening agent may have a molecular weight of at least about 100,000, or at least about 500,000 or at least about 1,000,000.

The composition may also optionally include at least one natural or synthetic rubber to impart impact resistance. Suitable rubbers are known to the skilled artisan. Such rubbers include, but are not limited to, dienes, styrenes, acrylonitriles, and mixtures thereof. Examples of suitable rubbers are disclosed in, for example, U.S. Pat. No. 4,313,865 and U.S. Pat. No. 4,560,723. Suitably, the composition contains from about 15 wt % to about 25 wt. % of the rubbers.

The composition may optionally also include one or more stabilizers, suitably both at least one anionic vapor phase stabilizer and at least one anionic liquid phase stabilizer. Suitably, each anionic vapor phase stabilizer is added to give a concentration of less than 200 parts per million (ppm). In certain embodiments, each anionic vapor phase stabilizer is present from about 1 to 200 ppm, suitably from about 10 to 75 ppm, for example from about 10 to 50 ppm. These stabilizing agents may inhibit premature polymerization. Suitable stabilizers may include those listed in U.S. Pat. No. 6,183,593.

The stability, and thus the shelf-life, of some monomeric adhesive compositions can be further enhanced and extended through careful regulation of the packaging. Treated (e.g., fluorinated polymer) packaging such as that disclosed in US-A-2003039781 may reduce the amount of stabilizer that is combined into the composition.

The compositions may also include pH modifiers in an amount effective to control the rate of degradation of the resulting polymer, as disclosed in U.S. Pat. No. 6,143,352.

Compositions of the present invention may also include at least one biocompatible agent effective to reduce active formaldehyde concentration levels produced during in vivo biodegradation of the polymer (also referred to herein as “formaldehyde concentration reducing agents”). Suitably, this component may be a formaldehyde scavenger compound. Examples of formaldehyde scavenger compounds useful in this invention include, but are not limited to sulfites; bisulfites; mixtures of sulfites and bisulfites, etc., as described in U.S. Pat. No. 5,328,687 or U.S. Pat. No. 5,624,669. The formaldehyde scavenger compound may be added in an amount effective to reduce the amount of formaldehyde released in vivo by the adhesive.

To improve the cohesive strength of adhesives formed from the compositions of this invention, difunctional monomeric cross-linking agents may be added to the monomer compositions of this invention. Such crosslinking agents are known. U.S. Pat. No. 3,940,362, discloses exemplary cross-linking agents. Suitably, from about 5 wt % to about 95 wt. % of the composition may be made up of the difunctional monomeric cross-linking agents, for example from about 20 wt. % to about 80 wt. % of the composition.

The compositions of this invention may further contain an effective amount of fibrous reinforcement and colorants such as dyes, pigments, and pigment dyes. Examples of suitable fibrous reinforcement include PGA microfibrils, collagen microfibrils, and others as described in U.S. Pat. No. 6,183,593.

In embodiments of the present invention, the composition and/or its applicator may contain materials such as a polymerization initiator, accelerator, rate-modifier, and/or cross-linking agent for initiating polymerization and/or cross-linking of the polymerizable monomer material. Suitable materials and applicators and packaging systems are disclosed in U.S. Pat. No. 5,928,611, U.S. Pat. No. 6,352,704 U.S. Pat. No. 6,455,064, WO-A-0132795, WO-A-0038777, WO-A-0132519, US-A-2003039781 and US-A-2003080151.

All weight percentages herein are based on the total weight of the α-cyanoacrylate adhesive composition.

Specific embodiments of adhesive compositions according to the invention will now be described further, by way of example, with reference to the accompanying drawings, in which:

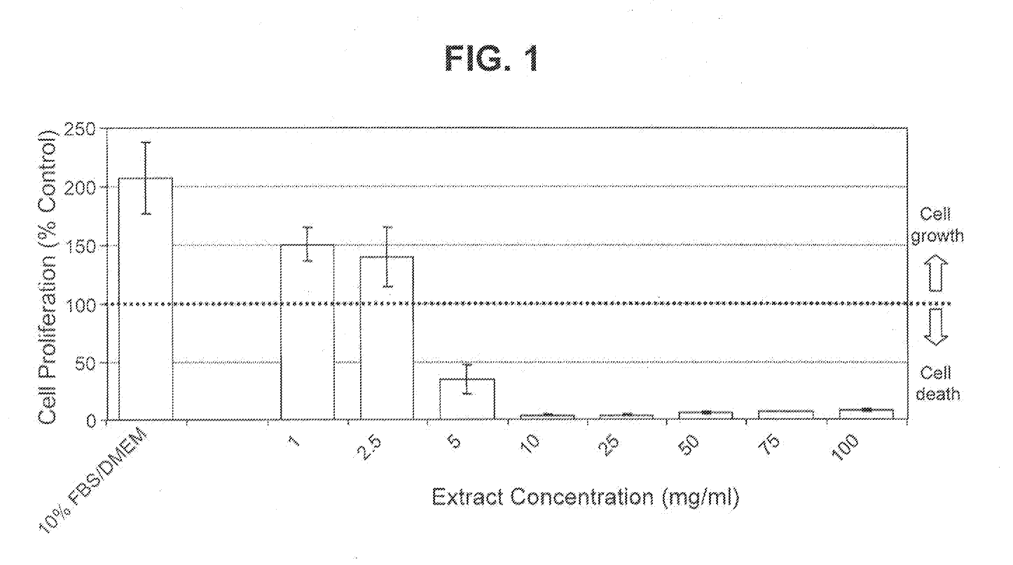

FIG. 1 shows a graph of measured fibroblast cell proliferation versus concentration of a primary serum extract for reference adhesives containing no NSAID;

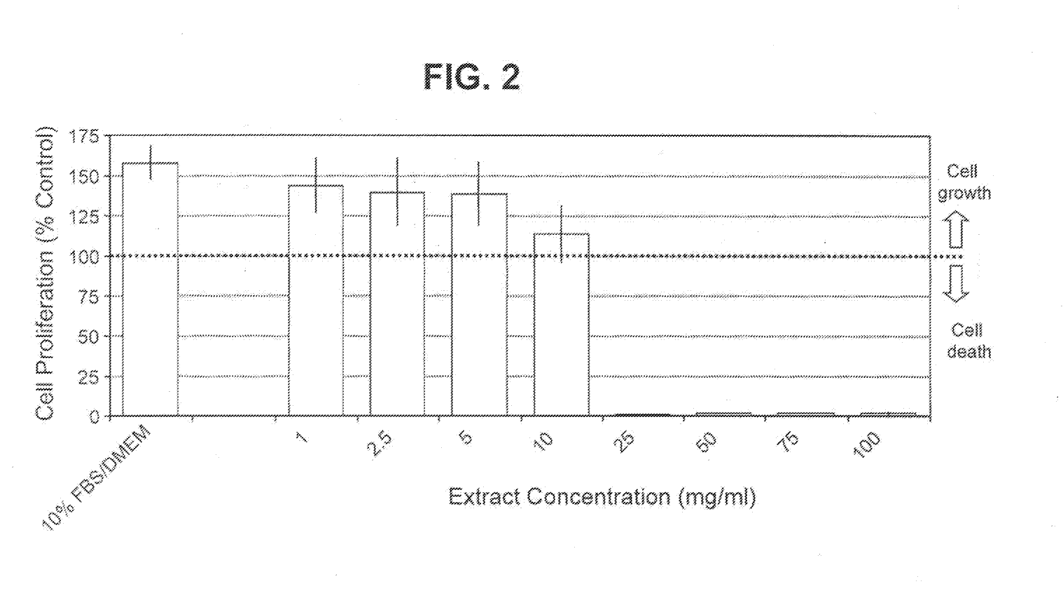

FIG. 2 shows a graph of measured fibroblast cell proliferation versus concentration of a primary serum extract for an adhesive composition according to the invention containing 1 wt. % of ibuprofen;

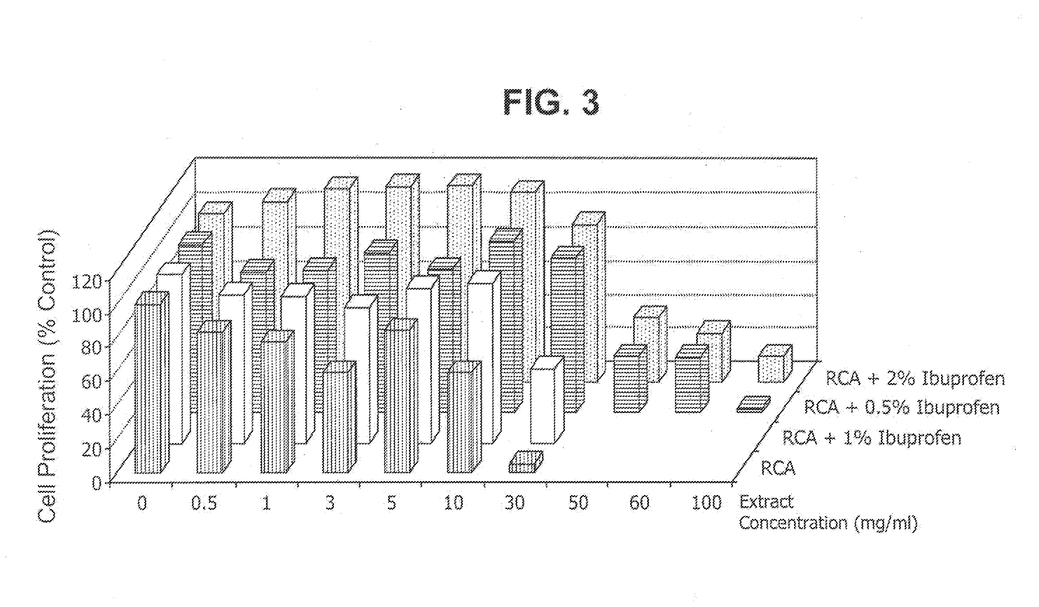

FIG. 3 shows a graph of measured fibroblast cell proliferation versus concentration for primary serum extracts from a series of resorbable cyanoacrylate adhesive (RCA) compositions containing 0% (control), 0.5 wt %, 1 wt. % and 2 wt. % of ibuprofen;

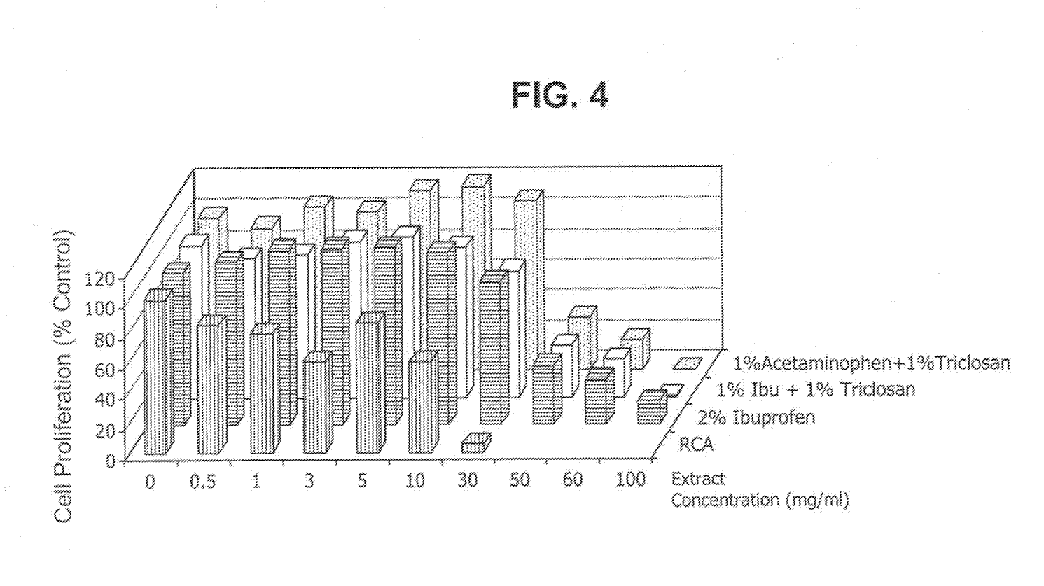

FIG. 4 shows a graph of measured fibroblast cell proliferation versus concentration for primary serum extracts from a series of resorbable cyanoacrylate adhesive (RCA) compositions containing 0% NSAID (control), 2 wt. % of ibuprofen, 1 wt. % Ibuprofen+1 wt. % Triclosan, and 1 wt. % Acetaminophen+1 wt. % Triclosan;

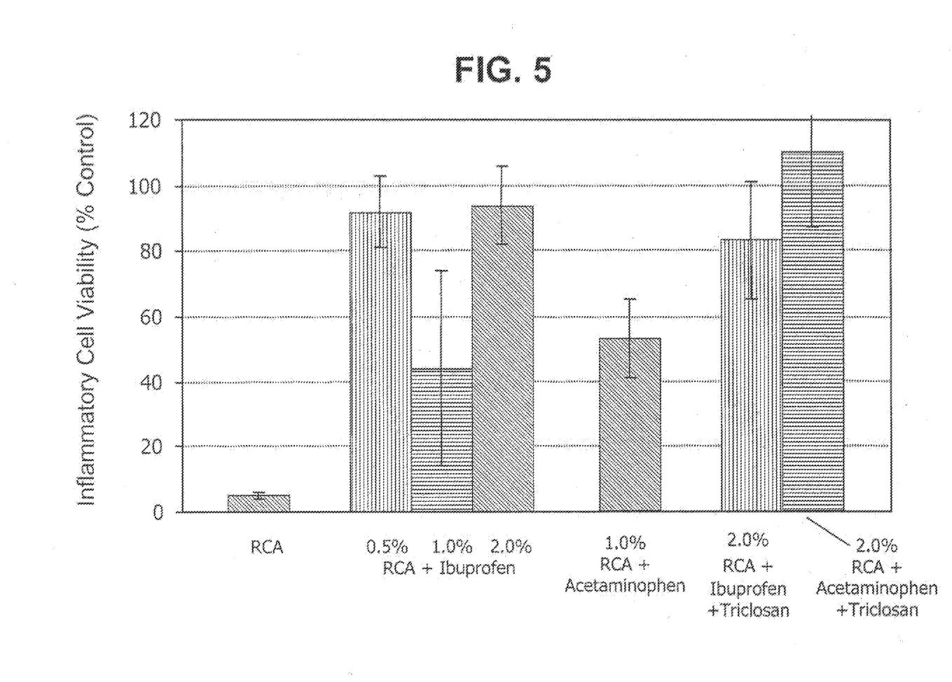

FIG. 5 shows a graph of measured inflammatory cell viability at a concentration of 10 mg/ml for primary extracts from a series of resorbable cyanoacrylate adhesive (RCA) compositions containing: 0% NSAID (control), 0.5 wt. %, 1.0 wt. % and 2 wt. % of Ibuprofen; 1.0 wt. % Acetaminophen; 1.0 wt. % Ibuprofen+1 wt. % Triclosan; and 1 wt. % Acetaminophen+1 wt. % Triclosan;

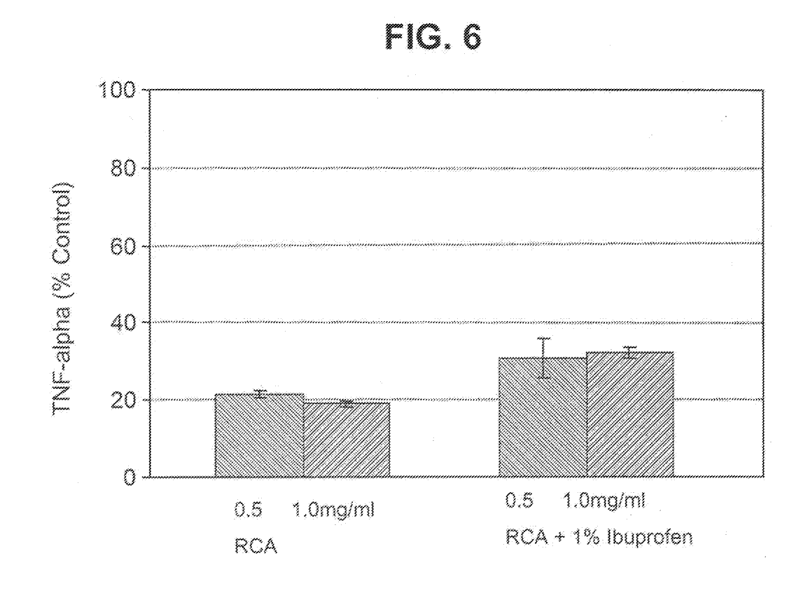

FIG. 6 shows a graph of measured TNF-a production by THP-1 inflammatory cells at concentrations of 0.5 and 1.0 mg/ml for primary serum extracts (10 mg/ml) from a series of resorbable cyanoacrylate adhesive (RCA) compositions containing: 0% (control), 0.5 wt. % and 1.0 wt. % of Ibuprofen;

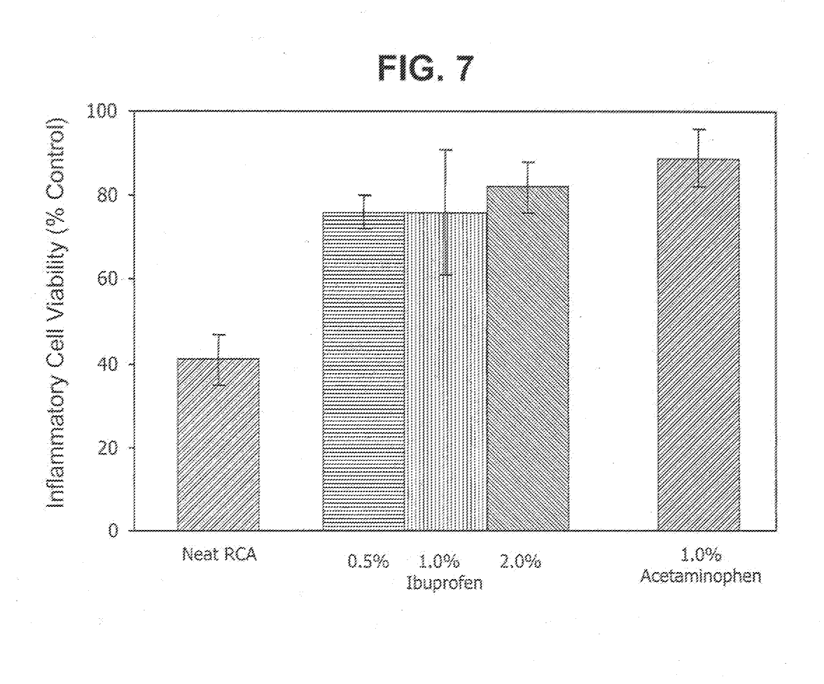

FIG. 7 shows a graph of measured THP-1 inflammatory cell viability for primary serum extracts (10 mg/ml) from a series of resorbable cyanoacrylate adhesive (RCA) compositions containing: 0% (control), 0.5 wt. % and 1.0 wt. % of Ibuprofen, and 1.0 wt. % of acetaminophen.

### EXAMPLE 1

A 0.5% solution of ibuprofen in 3-(2-cyano-acryloyloxy)-hexanoic acid ethyl ester (Et-β-CPL-CA) monomer was prepared by dissolving 12.5 mg of ibuprofen into 2.5 ml of the cyanoacrylate monomer into a glass vial. This solution was dispensed into five 1 ml acid treated and dried glass ampoules with 0.5 ml/ampoule. The overhead space of each ampoule was filled with 500 ppm SO₂-in-N₂ gas mixture and flame-sealed. The samples were sterilized by dry heat at 160° C. for 30 minutes. After sterilization, there was no visual change in viscosity and appearance of all samples.

### EXAMPLE 2

A 1.0% solution of ibuprofen in Et-β-CPL-CA monomer was prepared by dissolving 25.0 mg of ibuprofen into 2.5 ml of the cyanoacrylate monomer into a glass vial. This solution was dispensed into five 1 ml acid treated and dried glass ampoules with 0.5 ml/ampoule. The overhead space of each ampoule was filled with 500 ppm SO₂-in-N₂ gas mixture and flame-sealed. The samples were sterilized by dry heat at 160° C. for 30 minutes. After sterilization, there was no visual change in viscosity and appearance of all samples.

### EXAMPLE 3

A 2.0% solution of ibuprofen in Et-β-CPL-CA monomer was prepared by dissolving 50.0 mg of ibuprofen into 2.5 ml of the cyanoacrylate monomer into a glass vial. This solution was dispensed into five 1 ml acid treated and dried glass ampoules with 0.5 ml/ampoule. The overhead space of each ampoule was filled with 500 ppm SO₂-in-N₂ gas mixture and flame-sealed. The samples were sterilized by dry heat at 160° C. for 30 minutes. After sterilization, there was no visual change in viscosity and appearance of all samples.

### EXAMPLE 4

A 1.0% solution of acetaminophen in Et-β-CPL-CA monomer was prepared by dissolving 25.0 mg of acetaminophen into 2.5 ml of the cyanoacrylate monomer into a glass vial. This solution was dispensed into five 1 ml acid treated and dried glass ampoules with 0.5 ml/ampoule. The overhead space of each ampoule was filled with 500 ppm SO₂-in-N₂ gas mixture and flame-sealed. The samples were sterilized by dry heat at 160° C. for 30 minutes. After sterilization, there was no visual change in viscosity and appearance of all samples.

### EXAMPLE 5

A 2.0% solution of acetaminophen in Et-β-CPL-CA monomer was prepared by dissolving 50.0 mg of acetaminophen into 2.5 ml of the cyanoacrylate monomer into a glass vial. This solution was dispensed into five 1 ml acid treated and dried glass ampoules with 0.5 ml/ampoule. The overhead space of each ampoule was filled with 500 ppm SO₂-in-N₂ gas mixture and flame-sealed. The samples were sterilized by dry heat at 160° C. for 30 minutes. After sterilization, there was no visual change in viscosity and appearance of all samples.

### EXAMPLE 6

A solution of Et-β-CPL-CA monomer containing 1.0% ibuprofen and 1.0% triclosan was prepared by dissolving 25.0 mg of ibuprofen and 25.0 mg of triclosan into 2.5 ml of the cyanoacrylate monomer into a glass vial. This solution was dispensed into five 1 ml acid treated and dried glass ampoules with 0.5 ml/ampoule. The overhead space of each ampoule was filled with 500 ppm SO₂-in-N₂ gas mixture and flame-sealed. The samples were sterilized by dry heat at 160° C. for 30 minutes. After sterilization, there was no visual change in viscosity and appearance of all samples.

### EXAMPLE 7

A solution of Et-β-CPL-CA monomer containing 1.0% acetaminophen and 1.0% triclosan was prepared by dissolving 25.0 mg of acetaminophen and 25.0 mg of triclosan into 2.5 ml of the cyanoacrylate monomer into a glass vial. This solution was dispensed into five 1 ml acid treated and dried glass ampoules with 0.5 ml/ampoule. The overhead space of each ampoule was filled with 500 ppm SO₂-in-N₂ gas mixture and flame-sealed. The samples were sterilized by dry heat at 160° C. for 30 minutes. After sterilization, there was no visual change in viscosity and appearance of all samples.

### EXAMPLE 8

A 1% solution of ibuprofen in 2-octylcyanoacrylate (2-OCA) monomer was prepared by dissolving 10 mg of ibuprofen into 1 ml of the cyanoacrylate monomer into a 2 ml glass ampoule. The overhead space of the ampoule was filled with N₂ and flame-sealed. After 24 hours of storage at room temperature, there was no change in viscosity and appearance.

### EXAMPLE 9

A 1% solution of ketoprofen in 2-OCA monomer was prepared by dissolving 10 mg of ibuprofen into 1 ml of the cyanoacrylate monomer into a 2 ml glass ampoule. The overhead space of the ampoule was filled with N₂ and flame-sealed. After 24 hours of storage at room temperature, there was no change in viscosity and appearance.

### EXAMPLE 10

A 1% solution of curcumin in 2-OCA monomer was prepared by dissolving 10 mg of curcumin into 1 ml of the cyanoacrylate monomer into a 2 ml glass ampoule. The overhead space of the ampoule was filled with N₂ and flame-sealed. After 24 hours of storage at room temperature, there was no change in viscosity and appearance.

### EXAMPLE 11

A 1% solution of topiramate in 2-OCA monomer was prepared by dissolving 10 mg of topiramate into 1 ml of the cyanoacrylate monomer into a 2 ml glass ampoule. The overhead space of the ampoule was filled with N₂ and flame-sealed. After 24 hours of storage at room temperature, there was no change in viscosity and appearance.

The above examples have been described for the purpose of illustration only. Many other embodiments falling within the scope of the accompanying claims will be apparent to the skilled reader.

Procedure 1—Preparation of Extracts

A measured weight of the sealant to be tested was dispensed into tissue culture tube. The RCA base composition used in the preparation of these extracts was a 3-(2-cyano-acryloyloxy)-hexanoic acid ethyl ester (Et-b-CPL-CA) monomer with a purity of 99%. This resorbable cyanacrylate does not require the use of an activator. The sealants to be tested contained either no added NSAID (control), or 0.5 wt. % to 2 wt. % of dispersed NSAID (examples according to the invention) as specified further below. The sealant was then allowed to polymerize for about 5 minutes, after which serum-free cell culture medium was added (8 mls/tube). For fibroblast assays the culture medium was Dulbecco's modified Eagle's medium (DMEM). For the inflammatory cell assays, the culture medium was RPMI medium.

A primary extract was prepared by incubating the polymerized material with the medium for 24 hours, 37° C. If long-term effects of the material were being investigated, then a secondary extraction was performed by incubating the material with fresh medium for an additional 24 hours at 37° C. The medium containing the extract was then separated from the sealant for testing.

Extracts of different concentrations were prepared by varying the amount of the sealant to be tested in the above procedure. Thus, an extract with nominal concentration 10 mg/ml was prepared by dispensing 80 mg of the sealant into the tube as above, followed by polymerization and extraction with 8 ml of the serum.

Procedure 2—Fibroblast Growth/Viability Assay

Materials and Solutions
XTT, Cell Proliferation kit II, Cat no. 1465015, obtained from Boehringer Mannheim.
Adult Human Dermal Fibroblasts, Cat no. CRL-2465, supplied from American Type Culture Collection.
Phosphate Buffered Saline (PBS) Cat no. 14190-094, obtained from Life Technologies.
Dulbecco's Modified Eagles Medium (DMEM) Cat no. 31885-023 obtained from Life Technologies.
Fetal Bovine Serum (FBS) Cat no. 10084-077, from Life Technologies.
96-well microtitre plates, Cat no. 3072, from Becton Dickinson.

Optional—Standard PDGF-BB—human recombinant platelet derived growth factor PDGF-BB was obtained from R&D Systems.

Experimental Procedure

Adult Human Dermal Fibroblasts were harvested at 95% confluency and re-seeded in DMEM with 10% FBS at a cell density of 2.5×10⁴ cells/ml in a 96-well microtitre plate (100 μl/well). The cells were allowed to adhere and spread to the well surface for 24 hours in a humidified incubator, 37° C., 5% CO₂. The medium was then removed by aspiration and the cell monolayer washed with serum free DMEM.

Test samples or standards were added to the cell monolayer in serum free DMEM (100 μl/well); at least 4 replicates of each test sample/standard was tested. The standards used in this experiment were 10% FBS/DMEM, and serum free DMEM representing the normal growth pattern of dermal fibroblasts when maintained in nutritionally balanced medium versus a starvation medium. All samples were incubated with the cells for 72 hours at 37° C., 5% CO₂.

After this incubation time, the conditioned medium was removed and replaced with 100 μl serum free DMEM, then 50 μl of a labeling solution from the XTT cell proliferation kit was added to each well. Once this is added, an initial absorbance reading was obtained at 450 nm, after which, the microtitre plate was incubated at 37° C., 5% CO₂ and the absorbance monitored over 3 hours.

The effect of each test sample was evaluated by comparing the difference in absorbance readings measured against the standards.

The results of this procedure are shown in FIGS. 1 to 4. Referring to FIG. 1, which shows data for the control sample containing no added NSAID, it can be seen that the positive control 10% FBS/DMEM results in approximately 200% fibroblast proliferation, where 100% proliferation corresponds to the negative control sample of serum-free DMEM. The samples containing extracts from the resorbable cyanoacrylate adhesive (RCA) without any added NSAID exhibit show a sharp drop in cell proliferation for extract concentrations higher than about 5 mg/ml. This reflects the cytotoxicity of the adhesives towards fibroblasts.

Referring to FIGS. 2 to 4, these show data for samples of the same RCA containing 1 wt. % of ibuprofen (FIG. 2), for samples containing 0 wt. % (control), 0.5 wt. %, 1 wt. % and 2 wt. % of Ibuprofen (FIG. 3), and for samples containing 0 wt. % (control), 2 wt. % ibuprofen, 1 wt. % ibuprofen+1 wt. % triclosan, and 1 wt. % acetaminophen+1 wt. % triclosan (FIG. 4). it can be seen that the presence of the NSAIDs in the RCA results in maintenance of high levels of fibroblast cell proliferation at extract concentrations up to about 30 mg/ml. This illustrates the cytoprotective effect of the NSAIDs, in particular when combined with Triclosan.

Procedure 3—Inflammatory Cell Viability & Cytokine Release Assay

Materials and Solutions
RPMI 1640 Medium+2 mM Glutamine—obtained from GIBCO BRL, 500 ml, Cat Number 31095-029, stored at +4° C.
Antibiotic/Antimycotic solution (100×)—obtained from GIBCO BRL, Cat Number 15240-062. 10,000 U/ml penicillin, 10,000 μg/ml streptomycin and 20 μg/ml amphotericin B in 0.85% saline. Typically a 100 ml bottle is defrosted at room temperature (takes a few hours) and aliquoted (5 ml) into sterile centrifuge tubes under sterile conditions and stored frozen at −20° C. until required.
Fetal Calf Serum (FCS) or Fetal Bovine Serum (FBS) obtained from GIBCO BRL, 500 ml, Cat Number 10106-169, stored at −20° C. Typically, FCS/FBS (500 ml) is defrosted at room temperature overnight. Aliquots (50 ml) are then transferred to sterile flasks under sterile conditions and stored at −20° C. until required.
Standard growth medium—10% FBS (50 mls in 500 ml medium) in RPMI, 2 mM Glutamine+Antibiotic/antimycotic solution (5 mls in 500 ml medium)
PMA Solution (Phorbol 12-myristate 13-acetate)—supplied by SIGMA, Cat Number P8139, and quantity 1 mg. A 10 mM stock solution was prepared in DMSO and frozen (1 mg PMA in 162 uls DMSO). A 5×10⁻⁶M working solution of PMA was prepared by diluting the stock solution in SF-RPMI medium (10 ul in 20 mls medium). This working solution can again be aliquoted and frozen until needed (2.5 ml aliquots).
PMA Adherence medium—90 mls RPMI medium, 5 mls FBS, 5 mls working solution of PMA.
LPS Solution (Lipopolysaccharide from E Coli)—supplied by SIGMA, Cat Number L6529, reconstituted in PBS (Phosphate buffered saline) at 1 mg/ml, aliquoted & frozen (20 ul aliquots). A working solution (1 ug/ml) is prepared by diluting a 20 ul aliquot in 20 mls SF-RPMI medium.

Experimental Procedure

Inflammatory cells (THP-1 cells) were harvested by centrifugation (1000 rpm/10 mins) and re-suspended at a cell density of 1×10⁵ cells/ml PMA-adherence medium. This medium was prepared prior to use to limit the stress on the cells. Cells were plated in a 24-well plate, in this PMA-adherence medium and at a cell density of 1 ml/well (1×10⁵ cells/ml). The plate is then incubated for 48 hrs at 37° C. and in 5% CO₂. After this incubation period the medium, the cells were checked microscopically for adherence, and the medium was aspirated and replaced with 1 ml of test sample (extract)/negative control SF-RPMI medium/positive control LPS (1 ug/ml). The plate was then incubated for a further 24 hrs at 37° C. and in 5% CO₂. For each of the experiments according to Procedure 3 the concentration of the extract was 10 mg/ml.

The conditioned medium was then removed and stored frozen for cytokine analysis. Typically TNF-alpha ELISA (obtained from R&D systems) or Micro-array analysis was used to assess levels of inflammatory cytokines secreted by the cells within 24 hours. In addition cell viability was assessed on the remaining cell monolayer using either a trypan blue exclusion assay, or by measuring the metabolic activity of the remaining cells as estimated by the MTT assay (Supplied as a kit, manufacturer's instructions followed).

The results are shown in FIGS. 5 to 7 for extracts prepared as described in Procedure 1 above from 3-(2-cyano-acryloyloxy)-hexanoic acid ethyl ester (Et-b-CPL-CA) monomer. Referring to FIGS. 5 and 7, it can be seen that the control extract from the resorbable cyanoacrylate adhesive (RCA) containing no NSAID exhibited greatly reduced inflammatory cell viability, reflecting the toxicity of the RCA towards the THP-1 inflammatory cells. In contrast, the extracts from RCA samples containing NSAIDs and optionally also Triclosan in the amounts specified in FIGS. 5 to 7 exhibited greatly improved cell viability, thereby demonstrating that the NSAIDs have cytoprotective effect in these compositions. The cytoprotective effect is maintained, and may be enhanced, by the additional presence of Triclosan antimicrobial agent in the compositions.

Referring to FIG. 6, the data show that TNF-a production from THP-1 cells is maintained better in the presence of extracts of RCA containing Ibuprofen than in the presence of extracts without Ibuprofen. This further confirms that the activity of the inflammatory cells is protected by the presence of a NSAID in the RCA composition.

1. A medical adhesive composition comprising, based on the total weight of the composition, from about 50 wt. % to about 99.9 wt. % of one or more α-cyanoacrylate monomers and from about 0.1 wt. % to about 5 wt. % of one or more non-steroidal anti-inflammatory drugs (NSAIDs).

2. A medical adhesive composition according to claim 1, wherein the composition comprises, based on the total weight of the composition, from about 0.5 wt. % to about 2 wt. % of said one or more non-steroidal anti-inflammatory drugs (NSAIDs).

3. A medical adhesive composition according to any preceding claim, wherein said composition is biodegradable in vivo.

4. A medical adhesive composition according to any preceding claim, wherein said NSAID is selected from the group consisting of ibuprofen, acetaminophen, and mixtures thereof.

5. A medical adhesive composition according to any preceding claim, wherein said composition further comprises an antimicrobial agent.

6. A medical adhesive composition according to claim 5, wherein said antimicrobial agent comprises or consists essentially of Triclosan.

7. A medical adhesive composition according to any preceding claim, wherein said composition is sterile and sealed in an oxygen-impermeable container.

8. A kit comprising a first container that contains a medical adhesive composition according to claim 7 and a second container that contains a polymerization initiator or accelerator.

9. A medical adhesive polymer obtainable by polymerizing a medical adhesive composition according to any of claims 1 to 6.

10. A method of enhancing fibroblast cell viability in mammalian tissue in contact with an α-cyanoacrylate adhesive formed by polymerization of an α-cyanoacrylate adhesive composition applied to said tissue, said method comprising dispersing from about 0.1 wt. % to about 5 wt. %, based on the total weight of said composition applied to said tissue, of one or more non-steroidal anti-inflammatory drugs (NSAIDs) in said α-cyanoacrylate adhesive prior to application of said adhesive to said tissue.

11. A method of reducing cytotoxicity of an α-cyanoacrylate adhesive composition, said method comprising dispersing from about 0.1 wt. % to about 5 wt. %, based on the total weight of the composition, of one or more non-steroidal anti-inflammatory drugs (NSAIDs) in said α-cyanoacrylate adhesive prior to application of said adhesive to said tissue.

12. Use of a non-steroidal anti-inflammatory drug (NSAID) for the preparation of a medical adhesive composition comprising an α-cyanoacrylate monomer or polymer and from about 0.1 wt. % to about 5 wt. %, based on the total weight of the composition, of said NSAID for enhancing fibroblast cell viability in a mammalian tissue in contact with said medical adhesive.

13. Use of a non-steroidal anti-inflammatory drug (NSAID) for the preparation of a medical adhesive composition comprising an α-cyanoacrylate monomer or polymer and from about 0.1 wt. % to about 5 wt. %, based on the total weight of the composition, of said NSAID for reducing cytotoxicity of said medical adhesive composition.
